(19) United States
(12) Patent Application Publication
(10) Pub. No.: US 2024/0016631 A1
(43) Pub. Date: Jan. 18, 2024

(54) AN ENDOGRAFT DELIVERY DEVICE ASSEMBLY

(71) Applicant: Cook Medical Technologies LLC, Bloomington, IN (US)

(72) Inventors: James Collins, Queensland (AU); Logan Smith, Queensland (AU)

(73) Assignee: Cook Medical Technologies LLC, Bloomington, IN (US)

(21) Appl. No.: 18/350,471

(22) Filed: Jul. 11, 2023

(30) Foreign Application Priority Data
Jul. 12, 2022 (AU) 2022901946

(51) Int. Cl. A61F 2/95; A61F 2/07

(52) U.S. Cl. CPC A61F 2/9517; A61F 2/07; A61F 2002/061

(57) ABSTRACT

An endograft delivery device assembly a guide wire cannula extending distally from the tip to a handle assembly at the distal end. A positioner is disposed around the guide wire cannula. A sheath extends from the handle assembly over the positioner towards the tip. A sheath splitter assembly is within the handle assembly, and includes a splitter body attached to the positioner and disposed within the sheath and at least one splitter arm. The splitter body defines a through-hole through which a guide wire cannula passes. The splitter arm extends laterally from the splitter body though a slit though the sheath to a support within the handle assembly. The splitter body includes a circumferential sealing surface extending towards the tip, the sealing surface and the inner surface of the sheath dimensioned such that the sheath is circumferentially stretched over the sealing surface thereby forming a seal.

### RELATED APPLICATIONS

The present patent document claims the benefit of priority to Australian Patent Application No. 2022901946, filed Jul. 12, 2022, and entitled “An Endograft Delivery Device Assembly,” which is incorporated herein by reference.

### FIELD OF THE INVENTION

The present disclosure relates to medical devices. In particular, the present disclosure relates to endografts and their delivery systems, sometimes referred to as endoluminal delivery device assemblies or endograft delivery devices. More specifically, the present disclosure relates to endograft delivery device assemblies capable of delivering prostheses, endografts or stent grafts into the vascular system of humans or animals.

### BACKGROUND

The use of delivery devices or introducers employing catheters is known for a variety of medical procedures, including procedures for establishing, re-establishing or maintaining passages, cavities or lumens in vessels, organs or ducts in human and veterinary patients, occlusion of such vessels, delivering medical treatments, and other interventions.

Stent graft and delivery devices are used in aortic intervention. They are used by vascular surgeons to treat aneurysms and to repair regions of the aorta, including the aortic arch, the thoracic aorta, the abdominal aorta and the aortic bifurcation. Delivery devices allow deployment of intraluminal prostheses or endografts into the lumen of a patient from a remote location.

Numerous devises and procedures have been developed that involve the percutaneous insertion of a prosthesis into a body lumen, such as a blood vessel or duct, of a patient's body. Such a prosthesis may be introduced into the lumen by a variety of known techniques. For example, a wire guide may be introduced into a blood vessel using the Seldinger technique. This technique involves creating a surgical opening in the vessel with a needle and inserting a wire guide into the vessel through a bore of the needle. The needle can be withdrawn, leaving the wire guide in place. A delivery device is then inserted over the wire guide and into the vessel. The delivery device may be used in conventional fashion to insert into the blood vessel a variety of prostheses, such as stents, stent grafts, catheters, cardiac leads, balloons, and the like.

For example, the delivery device may be used to deliver and deploy an expandable prosthesis, such as a stent graft, to an aneurysmal blood vessel site. A stent graft is usually formed from a tubular body of a biocompatible graft material with one or more stents mounted into or onto the tubular body to provide support therefor. The stents may be balloon expandable stents and/or self-expanding stents. The deployment of the prosthesis into the lumen of a patient from a remote location by the use of an introducer delivery and deployment device is described in, for example, U.S. Pat. No. 7,435,253 to Hartley entitled “A Prosthesis and a Method and Means of Deploying a Prosthesis”, which is incorporated herein by reference herein in its entirety.

Endograft delivery devices typically include a seal and/or valve to retard or stop blood flow through their internal passages. Existing introducers and their seals/valves suffer a number of shortcomings. For instance, some leak blood more than is desirable.

It is commercially desirable to provide similar or the same delivery devices that can be used in many different procedures. This reduces inventory costs, simplifies supply chains and makes training of doctors easier. The provision of similar or the same delivery devices that can be used in multiple procedures is advantageous to operating staff, including doctors. It is also desirable to provide shorter and more efficient cannulation devices.

Generally, any side cannulation ports much be placed at a distance significantly distal of the seals and/or valves within the delivery device. This is because, with many existing delivery devices, a sheath, against which the seals engage, must be distally movable at least the equivalent distance to the length of retraction of the sheath required to deploy a prosthesis, such as a stent graft. Over this movable range, there cannot be any access points to within a positioner or pusher. The effect of this is that the overall length of the delivery device becomes quite long where devices employing side access ports are employed or are an optional add-on.

It is desirable in some applications to provide a sealed sheath that can be pulled past left and right access ports. Therefore, it is desirable to provide an improved endograft delivery device assembly that either allows this is or provides a universal design that facilitates creation of variants that allow this.

Throughout this specification, the term “distal” with respect to a portion of the aorta, a deployment device or an endograft means the end of the aorta, deployment device or endograft further away in the direction of blood flow from the heart and the term “proximal” means the portion of the aorta deployment device or end of the endograft nearer to the heart in the direction of blood flow.

### SUMMARY

Disclosed is an endograft delivery device assembly having a proximal end, a distal end and a prosthesis receiving zone, a tip at the proximal end, a guide wire cannula extending distally from the tip to a handle assembly at the distal end and being slidable over a guide wire. A positioner is disposed around the guide wire cannula, and extends from the handle assembly towards the tip. A sheath extends from the handle assembly over the positioner towards the tip and has an inner surface. A sheath splitter assembly is within the handle assembly, and includes a splitter body attached to the positioner and disposed within the sheath and at least one splitter arm. The splitter body defines a through-hole through which the guide wire cannula passes. The at least one splitter arm extends laterally from the splitter body though a slit though the sheath to a support within the handle assembly. The splitter body includes a circumferential sealing surface extending towards the tip, the sealing surface and the inner surface of the sheath dimensioned such that the sheath is circumferentially stretched over the sealing surface thereby forming a seal.

The sheath may include a prosthesis sheathing portion, the prosthesis sheathing portion moveable from a covering position in which it covers the prosthesis receiving zone and a retracted position in which the prosthesis sheathing portion is longitudinally displaced way from the prosthesis receiving zone. The splitter body may include a ramp portion, in use, the ramp portion providing a gradual stretching of the sheath as it is pulled distally away from the tip and into the handle assembly. The ramp portion may include a pair of projections extending toward the tip.

The splitter body may include a pair of arms, each arm supported by the support within the handle assembly. The assembly may further comprise a follower attached to the sheath, the follower drivable relative to the sheath splitter assembly in a direction away from the proximal end of the delivery device assembly, where movement of the follower in a direction away from the proximal end of the delivery device assembly slides the sheath over the sealing surface and pulls the prosthesis sheathing portion of the sheath from the covering position in which it covers the prosthesis receiving zone to the retracted position in which the prosthesis sheathing portion is longitudinally displaced way from the prosthesis receiving zone. The follower may be attached to the sheath at sheath distal end, and wherein the follower is sandwiched between a pair of spaced apart sheath portions.

The assembly may further include a stiffener disposed around the guide wire cannula, the stiffener extending distally from the splitter body. The stiffener is supported at a distal end thereof by the handle assembly.

The arm, or each arm, may include a side access passage into the through-hole of the splitter body. The side access passage may include a side seal, the side seal arranged and constructed to sealably receive a cannula. The side access passage(s) extend out through the main grip portion of the handle assembly, thereby allowing cannulation of side fenestrations within an endograft. The arm, or each arm, may present an angled edge, with respect to a plane orthogonal to the positioner, to the sheath.

Also disclosed is an endograft delivery device assembly including a proximal end, a distal end, a prosthesis receiving zone, a tip at the proximal end, a handle assembly at the distal end, a guide wire cannula extending distally from the tip to the handle assembly, the guide wire cannula configured to slidably receive a guide wire, a multi lumen positioner disposed around the guide wire cannula, the multi lumen positioner extending from the handle assembly towards the tip, wherein the handle assembly is configured to accommodate the multi lumen positioner, a sheath extending from the handle assembly over the positioner towards the tip, the sheath having an inner surface, wherein the sheath includes a prosthesis sheathing portion, the prosthesis sheathing portion moveable from a covering position in which it covers the prosthesis receiving zone and a retracted position in which the prosthesis sheathing portion is longitudinally displaced way from the prosthesis receiving zone, and a sheath splitter assembly within the handle assembly. The sheath splitter assembly includes a splitter body attached to the positioner and disposed within the sheath, the splitter body defining a through-hole through which the guide wire cannula passes, and at least one splitter arm, the at least one splitter arm extending from the splitter body though a slit though the sheath to a support within the handle assembly, wherein the at least one splitter arm presents an angled edge, with respect to a plane orthogonal to the positioner, to the sheath, wherein the splitter body includes a circumferential sealing surface extending towards the tip, the sealing surface and the inner surface of the sheath dimensioned such that the sheath is circumferentially stretched over the sealing surface thereby forming a seal.

The assembly may include splitter arm mounts holding the at least one splitter arm in position relative to the handle assembly. The least one splitter arm may have a side seal disposed within the at least one splitter arms, the side seal configured to prevent blood loss through the positioner.

The assembly may include a follower attached to the sheath, the follower drivable relative to the sheath splitter assembly in a direction away from the proximal end of the delivery device assembly. Movement of the follower in a direction away from the proximal end of the delivery device assembly slides the sheath over the sealing surface and pulls the prosthesis sheathing portion of the sheath from the covering position in which it covers the prosthesis receiving zone to the retracted position in which the prosthesis sheathing portion is longitudinally displaced way from the prosthesis receiving zone. The follower is operatively attached to the sheath at a sheath distal end, and wherein the follower is sandwiched between a pair of spaced apart sheath portions.

Further disclosed is an endograft delivery device assembly including a proximal end, a distal end, a prosthesis receiving zone, a tip at the proximal end, a handle assembly at the distal end, a guide wire cannula extending distally from the tip to the handle assembly, the guide wire cannula configured to slidably receive a guide wire, a positioner disposed around the guide wire cannula, the positioner extending from the handle assembly towards the tip, a sheath extending from the handle assembly over the positioner towards the tip, the sheath having an inner surface, wherein the sheath includes a prosthesis sheathing portion, the prosthesis sheathing portion moveable from a covering position in which it covers the prosthesis receiving zone and a retracted position in which the prosthesis sheathing portion is longitudinally displaced way from the prosthesis receiving zone, and

a sheath splitter assembly within the handle assembly. The sheath splitter assembly includes a splitter body attached to the positioner and disposed within the sheath, the splitter body defining a through-hole through which the guide wire cannula passes, and at least one splitter arm having a side access passage into the through-hole of the splitter body, the at least one splitter arm extending from the splitter body though a slit though the sheath to a support within the handle assembly. The splitter body includes a circumferential sealing surface extending towards the tip, the sealing surface and the inner surface of the sheath dimensioned such that the sheath is circumferentially stretched over the sealing surface thereby forming a seal. The at least one splitter arm comprises a side seal disposed within the at least one splitter arm, the side seal configured to prevent blood loss through the positioner. The sheath is configured to be pulled past the side access passage without breaking a hemostatic seal

### BRIEF DESCRIPTION OF DRAWINGS

Embodiments of the present disclosure will be discussed with reference to the accompanying drawings wherein:

FIG. 1A shows an endograft delivery device assembly according to the disclosure in an isometric view;

FIG. 1B is a similar view to that of FIG. 1A and shows a sheath retracted to expose a stent graft;

FIG. 2 is a cutaway isometric view of a portion of the endograft delivery device shown in FIGS. 1A and 1B;

FIG. 3 is a cutaway and exploded view of a portion of the endograft delivery device of FIGS. 1A, 1B and 2;

FIG. 4 is an isometric view of a sheath splitter assembly component of the endograft delivery device of FIGS. 1 and 1B;

FIG. 5 is a similar view to that of FIG. 4, but shows the sheath splitter assembly from an alternative viewing angle;

FIG. 6 is a top view of the sheath splitter assembly of FIGS. 4 and 5;

FIG. 7 is an end view of the sheath splitter assembly of FIGS. 4 and 5;

FIG. 8 is a sectional view taken along section lines A-A of the sheath splitter assembly shown in FIG. 6;

FIG. 9 is an isometric view of a sheath component being split over the sheath splitter assembly of FIGS. 4 and 5;

FIG. 10 is a sectional isometric view of a portion of the endograft delivery device assembly of FIGS. 1A and 1B;

FIG. 11 is an isometric view of an alternative embodiment of the sheath splitter assembly shown in FIGS. 4 and 5;

FIG. 12 is a top view of the alternative sheath splitter assembly shown in FIG. 11;

FIG. 13 is a partial sectional view taken through section lines 13-13 indicated on FIG. 12;

FIG. 14 is an isometric view similar to that of FIGS. 1A and 1B, but shows an alternative endograft delivery device assembly having the sheath splitter assembly of FIGS. 11 to 13 to facilitate left and right side cannula access;

FIG. 15 is an isometric view of the sheath splitter assembly shown in FIG. 11 with further components added;

FIG. 16 is a side view of the sheath splitter assembly of FIG. 15;

FIG. 17 is a plan view of the sheath splitter assembly of FIG. 15;

FIG. 18 is an isometric sectional view through session lines 17-17 as indicated on FIG. 16; and

FIG. 19 is a cutaway isometric view similar to that of FIG. 3, but showing the alternative embodiment illustrated in FIGS. 11 to 18.

### DESCRIPTION

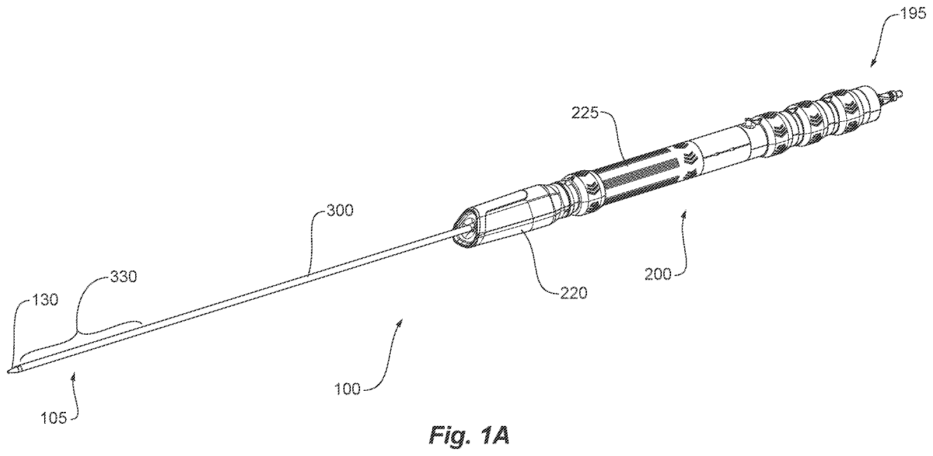

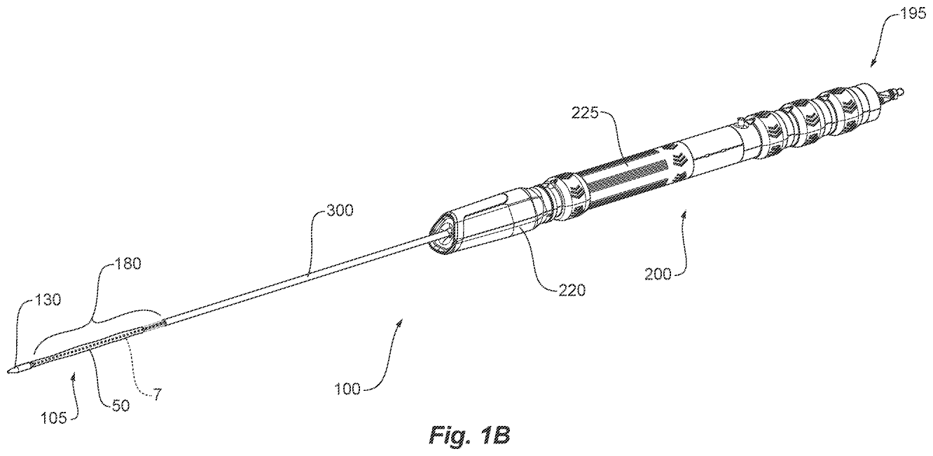

Referring to FIGS. 1A and 1B, an endograft delivery device assembly 100 is shown. The assembly device 100 has a proximal end 105, a distal end 195 and a prosthesis receiving zone 180. The prosthesis receiving zone 180 is shown in FIG. 1B. In FIG. 1A, the prosthesis receiving zone 180 is covered by a sheath 300. The delivery device assembly 100 further comprises a tip 130 at the proximal end, a guide wire cannula 7, shown in FIG. 1B, extending distally from the tip 130 to a handle assembly 200 at the distal end 195. The guide wire cannula 7 is slidable over a guide wire 4. The handle assembly 200 has a main grip portion 220. A positioner 400, disposed around the guide wire cannula 7 is illustrated in FIG. 10. The positioner 400 extends from the handle assembly 200 towards the tip 130. The sheath 300 extends from the handle assembly 200 over the positioner 400 towards the tip 130.

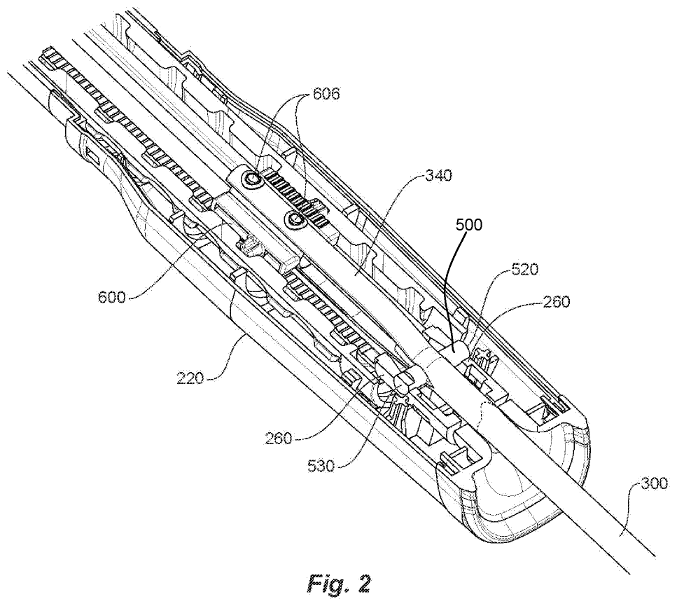

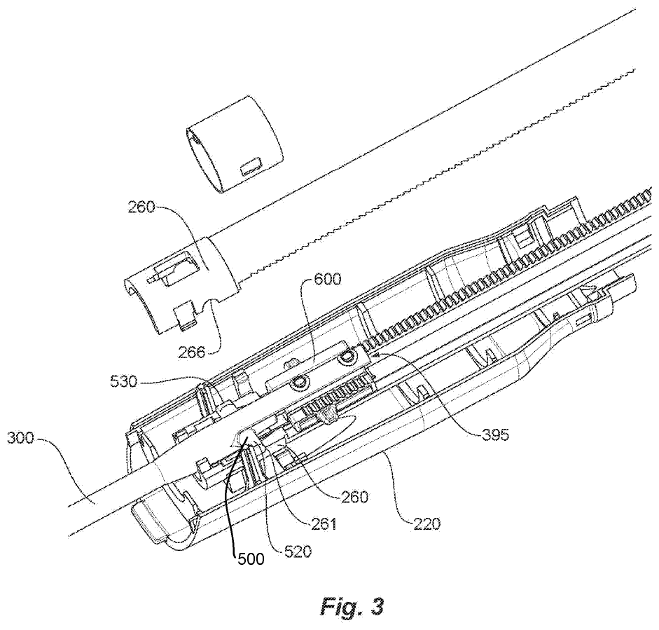

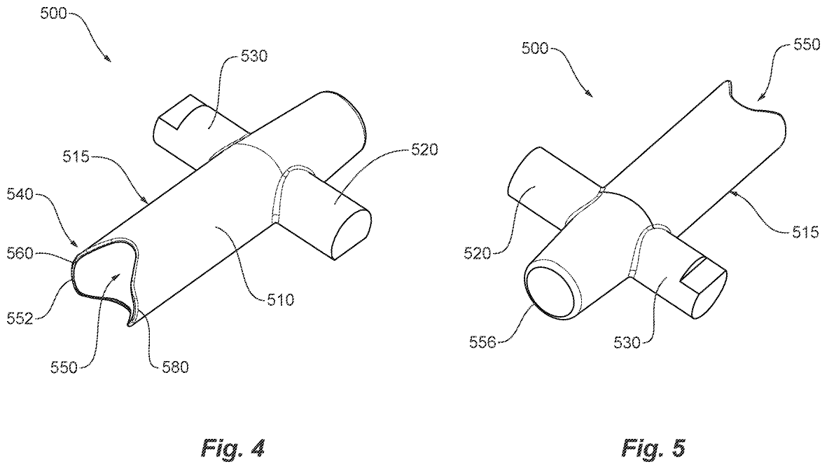

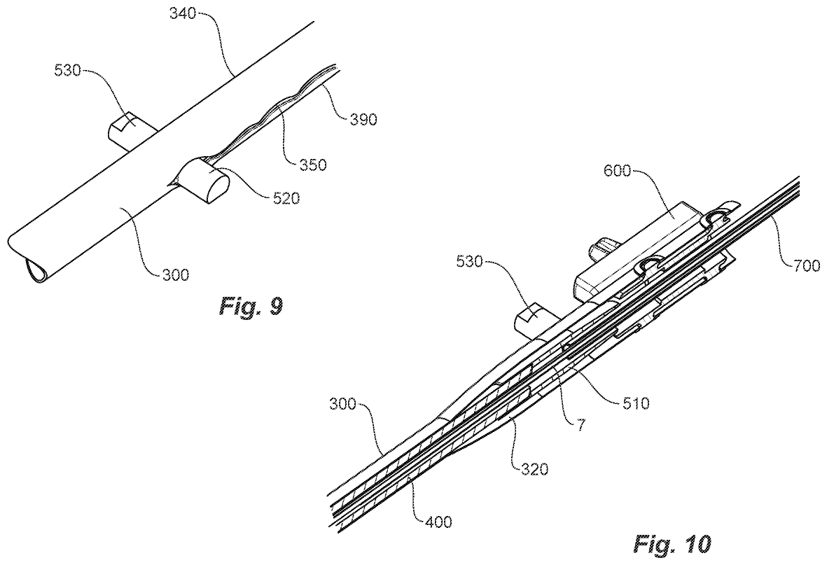

Referring now to FIGS. 2, 3, 4 and 5, a sheath splitter assembly 500 within the handle assembly 200 is shown. The sheath splitter assembly includes: a splitter body 510 which is most clearly shown in FIGS. 4 and 5. The splitter body 510 is attached to the positioner 400 and disposed within the sheath 300 as is shown in FIG. 10. The splitter body 510 defines a through-hole 550 through which the guide wire cannula 7 passes. This through-hole 550 is again shown most clearly in FIGS. 4 and 5. The sheath splitter assembly 500 further includes at least one splitter arm 520. As shown in FIG. 9, the splitter arm or arms 520, 530 extend laterally from the splitter body 510 through a slit 350 through the sheath 300 to a support 260 within the handle assembly 200. The support 260 is shown most clearly in FIGS. 2 and 3. In FIG. 2, a first support portion 261 is shown and in FIG. 3 a second support portion 266 is shown. The first and second support portions 261, 266 are shaped to snugly receive and support splitter arms 520, 530 when the support 260 is assembled. The splitter body 510 includes a circumferential sealing surface 515 extending towards the tip, as is illustrated in FIGS. 4 and 5. The sealing surface 515 and the inner surface 320 of the sheath 300 are dimensions such that the sheath 300 circumferentially stretches over the sealing surface 515 thereby forming a seal. The seal is a hemostatic seal that arrests bleeding.

Referring to FIG. 4, a proximal end mouth 550 of the splitter body 510 is shown. The positioner 400 nests inside this mouth 550 as can be seen in FIG. 10. This provides structural support for the positioner 400 via the splitter assembly 500, the splitter assembly being supported by splitter arms 520, 530, as described above and by a stiffener 700 as will be described below.

Referring now to FIGS. 9 and 10, details of internal components of the endograft delivery device assembly 100 are shown. A stiffener 700, shown in FIG. 10, is disposed around the guide wire cannula 7. The stiffener 700 extends distally from the splitter body 510. It nests inside a distal end mouth 556 of the splitter body 510, the proximal end mouth 550 shown in FIG. 5.

The stiffener may not be required for some embodiments of the invention as the splitter arms 520, 530 are received and supported within the handle assembly 200 as explained above. However, as illustrated in FIGS. 2, 3, 4 and 5, the stiffener provides additional support that assists in stabilizing and supporting the sheath splitter assembly 500 within the handle assembly 200, including against loads imparted during the pull back of the sheath 300. Typically, where a stiffener is employed, such as stiffener 700 shown in FIG. 10, its distal end will be supported by the handle assembly 200 toward its distal end, allowing it to transmit loads, including compressive loads, from the spiller assembly 500 back to the handle assembly 200 without problematic bending or other misalignments that could otherwise cause jamming of the follower 600.

Returning to FIGS. 1 and 2, it can be seen that the sheath 300 includes a prosthesis sheathing portion 330. The prosthesis sheathing portion 330 is movable from a covering position, shown in FIG. 1A, in which it covers the prosthesis receiving zone 180 and a retracted position in which the prosthesis sheathing portion 330 is longitudinally displaced away from the prosthesis receiving zone 180 as is illustrated in FIG. 1B.

The sheath splitter assembly 500 shown in FIGS. 4 and 5 will now be discussed in further detail. The splitter body 510 includes a ramp portion 540. In use, the ramp portion 540 provides a gradual stretching of the sheath 300 as it is pulled distally away from the tip 130 and into the handle assembly 200. While various ramp portions may be used, with the embodiment illustrated in FIGS. 4 and 5, the ramp portion 540 includes a pair of projections 560, 580 extending towards the tip 130. As shown, the splitter body 510 includes a pair of arms 520, 530, each arm supported by the support 260 within the handle assembly 200 as is shown in FIGS. 2 and 3.

As shown in FIGS. 2 and 3, the endograft delivery device assembly 100 also includes a sheath follower 600 attached to the sheath 300. The follower 600 is driveable relative to the sheath splitter assembly 500 in a direction away from the proximal end 105 of the delivery device assembly 100. The follower 600 is driven by a hand-rotatable sheath retraction actuator ring 225. Movement of the follower 600 in a direction away from the proximal end 105 of the delivery device assembly 100 slides the sheath 300 over the sealing surface 515 and pulls the prosthesis sheathing portion 330 of the sheath 300 from the covering position shown in FIG. 1A in which it covers the prosthesis receiving zone 180, to the retracted position shown in FIG. 1B in which the prosthesis sheathing portion 330 is longitudinally displaced away from the prosthesis receiving zone 180.

Returning to FIGS. 2 and 3, the follower 600 is attached to the sheath 300 at a distal sheath end 395. The follower 600 is sandwiched between a pair of spaced apart sheath portions 340, 390. These sheath portions 340, 390 are perhaps best seen in FIG. 9.

Referring to FIGS. 2, 3, 9 and 10, it can be seen that the sheath 300 has a proximal portion that forms a continuous tube around the positioner 400 and a distal portion that is split into sheath portion halves 340, 390. As the sheath 300 is withdrawn, the slit 350 lengthens. However, the delivery device assembly 100 is constructed such that the slit 350 always remains distal relative to the sealing surface 515. This ensures that there is no leakage through the slit 350. The sheath 300 is compliant which enables a hemostatic seal to be maintained with a low pullback force that is readily induced by rotation of the handle 215. The sealing surface 515 and the seal created between the sealing surface 515 and the inner surface 320 of the sheath 300 remain stationary relative to the handle assembly 200 throughout use in delivering a prosthesis such as an endograft 50. This isolates the seal from bending and twisting movements that would usually be associated with moving followers such as the follower 600.

The sheath splitter assembly 500 within the handle assembly 200, in addition to facilitating the splitting of the sheath 300, assists in preventing the transmission or build-up of twisting forces due to the splitter body 510 having arms that are constrained within recesses or holes moulded into the supports 260 as is shown in FIGS. 2 and 3. The supports 260 may form part of a pair of spaced-apart toothed rails as is shown in FIGS. 2 and 3. Other constructions may also be used.

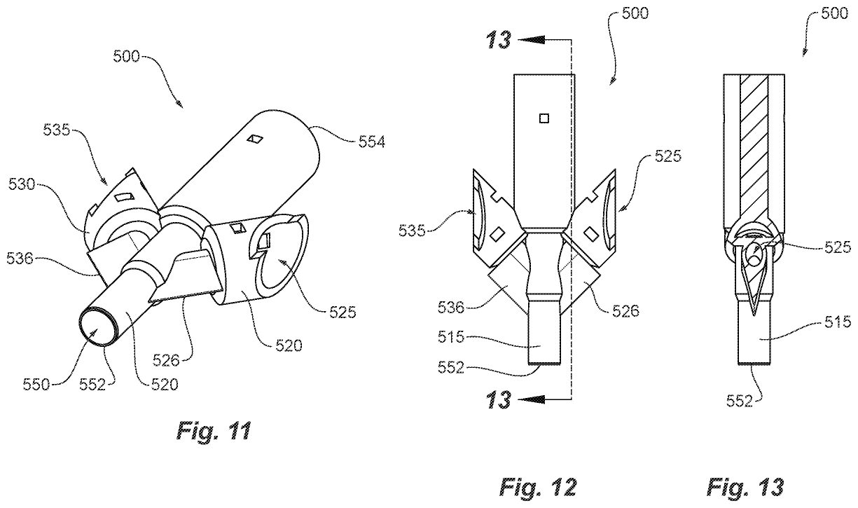

Referring now to FIGS. 11 to 19, a second embodiment of the disclosure is shown. Here, the sheath splitter assembly 500 within the handle assembly 200 is arranged and constructed to accommodate a multi-lumen positioner or pusher. Referring to FIGS. 11, 12 and 13, the sheath splitter assembly 500 shown has arms 520 and 530 that each include a side access passage 525, 535 into the through-hole 550 of the splitter body 510. The splitter arms 520 and 530 include splitter edges 526 and 536 that assist in splitting the sheath 300. The splitter edges 526 and 536 are angled with respect to a plane orthogonal to the positioner 400.

With this second embodiment illustrated in FIGS. 11 to 13, a sealing surface is provided. This sealing surface 515 engages with the inner surface 320 of the sheath 300 to create a seal. As the sheath 300 is pulled back over the sealing surface 515, under the action of the driven follower 600, it stretches thereby providing sealing engagement with the sealing surface 515. While not shown in FIGS. 11 to 13, a ramp portion may be provided. The ramp portion may take any suitable form for instance. One suitable form may be similar to the ramp portion 540 shown in FIGS. 4 and 5.

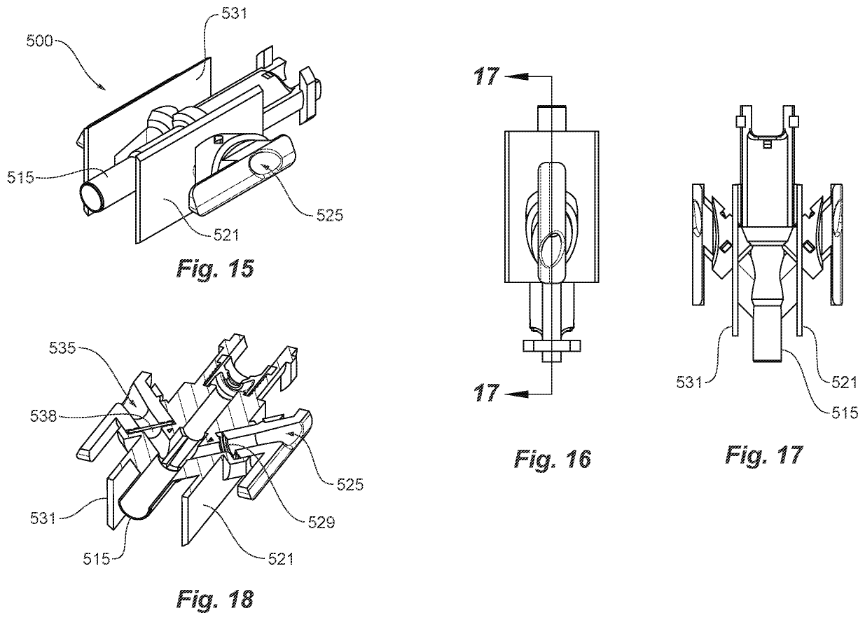

Referring now to FIGS. 15, 16, 17 and 18, the sheath splitter assembly 500 of the second embodiment will be described in more detail. FIG. 15 shows splitter arm mounts 521 and 531. These arm mounts 521, 531 hold the splitter arms 520, 530 in position within the handle assembly 200.

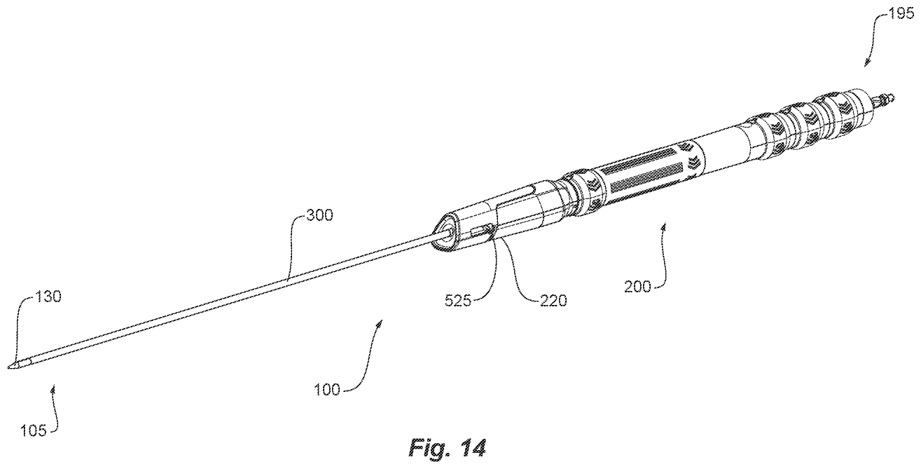

Turning now to FIG. 16, a sectional line 17-17 is shown. FIG. 18 is a cutaway isometric view through section lines 17-17 of FIG. 16. In FIG. 18, the internals of the passages 525 and 535 within the splitter arms 520 and 530 respectively are shown. One of the two side seals is also illustrated. Specifically, side seal 538 is shown in FIG. 18. A similar or identical side seal would typically also be provided in the splitter arm 520. However, this seal is omitted so as to show the side seal mounting groove 529 provided for the side seal 528. The side seals 528 and 538 are arranged and constructed to prevent blood loss that would otherwise occur through the positioner 400. The side seals 528 and 538 also allow side access cannulas to enter in through the handle, as shown in FIG. 14, through the seals 528 and 538, along inside the positioner 400 and then exiting at the prosthesis receiving zone 180 illustrated in FIG. 1B.

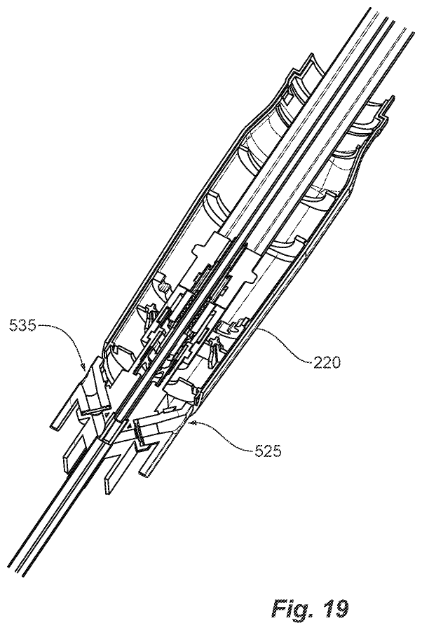

Referring to FIGS. 14 and 19, the position of the second embodiment of the sheath splitter assembly 500 is shown within the handle assembly 200. It can be seen that the sealed side access ports or passages 525, 535 are in a forward, or proximal, position which minimises the cannulation lengths required for fenestrations within endografts 50. The sealed sheath 300 is able to be pulled past these side access passages 525, 535 without breaking a hemostatic seal.

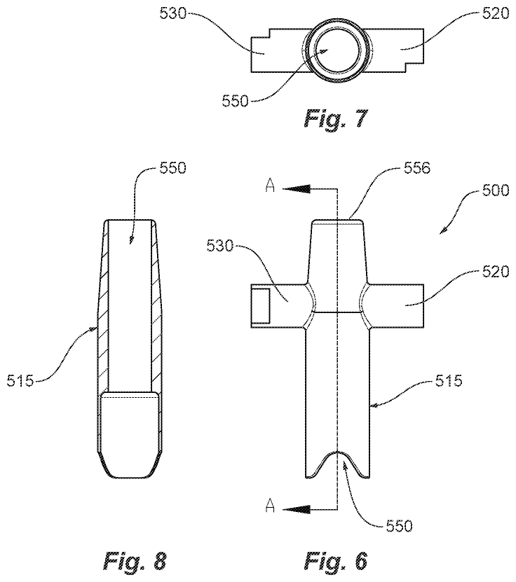

Use of a first example of a delivery device assembly 100 during a procedure will now be described in brief with reference to FIGS. 1A to 18. The delivery device assembly 100 is tracked to a desired location within a patient's body. At this stage, the prosthesis in the form of an endograft 50 is located at the proximal end 105 of the delivery device assembly 100 is fully covered by the sheath 300 and held in a radially inwardly compressed condition. To deploy the endograft 50 in a vessel lumen, withdrawal of the sheath 300 is induced by manipulations of the handle assembly 200.

In one example, a surgeon may retract the sheath 300 and expose the prosthesis as shown in the progression from FIGS. 1A to 1B. This may be achieved by rotation of the sheath retraction actuator ring 225 of the handle assembly 200, which drives the follower 600 and its sheath engaging projections 606, 608 to pull the sheath portions 340, 390 into the handle assembly 200. As this occurs, the inner surface 320 of the sheath 300 slides past the sealing surface 515 while maintaining a hemostatic seal, while behind the sealing surface the slit 350 lengthens as the follower moves distally along within the handle assembly 200.

Referring now to FIGS. 11 to 19, use of a second example of a delivery device assembly 100 will now be described in brief. The delivery device assembly 100 of FIGS. 14 and 19 can be used to deliver an endograft 50 to a vessel having a branch vessel extending from a main vessel where cannulation of the branch vessel is necessary or desired.

The sheath 300 may be only partially retracted from the endograft 50 to expose a fenestration (not shown) in the endograft 50 for cannulation of the branch vessel through the fenestration prior to full deployment of the endograft 50. This may provide a higher degree of control over the position of the endograft 50 and delivery device assembly 100 during the cannulation procedure. Once cannulation of the branch vessel is complete, the sheath 300 may be retracted further until it is fully removed from the endograft 50. Alternatively, the sheath 300 may be fully removed from the endograft 50 prior to cannulation.

In one example of a procedure using either of the two above-described examples, when the sheath 300 has been retracted a sufficient distance to expose a proximal end of the endograft 50 and before retracting the sheath 300 sufficiently distally to expose a distal end of the endograft 50, the user may proceed with removal of at least proximal trigger wires and any diameter reducing ties that may be present at the proximal end of the endograft 50 (such as by manipulation of one or more trigger wire release mechanisms of the handle assembly 200 to retract the proximal trigger wires) to allow the proximal end of the endograft 50 to at least partially deploy radially outwardly within a vessel. Alternatively, the user may proceed with removal of at least the proximal trigger wires and any other diameter reducing ties that may be present at the proximal end of the endograft 50 after the sheath 300 has been retracted a sufficient distance to expose the proximal end of the endograft 50 and the sheath 300 has been retracted distally to expose the distal end of the endograft 50. Exemplary diameter reducing tie systems and trigger wires are disclosed in US Publication No. 2007-0043425, “Assembly of Stent Grafts,” which is incorporated herein by reference in its entirety.

When the sheath 300 has been sufficiently retracted to expose the distal end of the endograft 50, the user may then retract the distal trigger wires and/or any other diameter reducing ties that may be present at the distal end of the endograft 50 (such as by manipulation of one or more trigger wire release mechanisms of the handle assembly 200 to allow the distal end of the endograft 50 to at least partially deploy radially outwardly within a vessel. At this point, the endograft 50 may be fully deployed within the vessel. In another example, the handle assembly 200 may be manipulated further to facilitate release of other components, for example a top cap (not shown) to allow the endograft 50 to fully deploy. Once the endograft 50 has been fully released from the delivery device assembly 100, the delivery device assembly 100 can be removed from the patient's body.

Throughout this specification and the claims that follow unless the context requires otherwise, the words ‘comprise’ and ‘include’ and variations such as ‘comprising’ and ‘including’ will be understood to imply the inclusion of a stated integer or group of integers but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims. Further, the features of any described example may be included as features of the other described examples.

1. An endograft delivery device assembly comprising:
a proximal end;
a distal end;
a prosthesis receiving zone;
a tip at the proximal end;
a handle assembly at the distal end;
a guide wire cannula extending distally from the tip to the handle assembly, the guide wire cannula configured to slidably receive a guide wire;
a positioner disposed around the guide wire cannula, the positioner extending from the handle assembly towards the tip;
a sheath extending from the handle assembly over the positioner towards the tip, the sheath having an inner surface;
a sheath splitter assembly within the handle assembly, the sheath splitter assembly including:
a splitter body attached to the positioner and disposed within the sheath, the splitter body defining a through-hole through which the guide wire cannula passes; and
at least one splitter arm, the at least one splitter arm extending laterally from the splitter body though a slit though the sheath to a support within the handle assembly,
wherein the splitter body includes a circumferential sealing surface extending towards the tip, the sealing surface and the inner surface of the sheath dimensioned such that the sheath is circumferentially stretched over the sealing surface thereby forming a seal.

2. The endograft delivery device assembly of claim 1, wherein the sheath includes a prosthesis sheathing portion, the prosthesis sheathing portion moveable from a covering position in which it covers the prosthesis receiving zone and a retracted position in which the prosthesis sheathing portion is longitudinally displaced way from the prosthesis receiving zone.

3. The endograft delivery device assembly of claim 1, wherein the splitter body includes a ramp portion configured to provide, in use, a gradual stretching of the sheath as it is pulled distally away from the tip and into the handle assembly.

4. The endograft delivery device assembly of claim 2, wherein the ramp portion includes a pair of projections extending toward the tip.

5. The endograft delivery device assembly of claim 1, wherein the at least one splitter arm comprises a pair of arms, each arm of the pair of arms supported by the support within the handle assembly.

6. The endograft delivery device assembly of claim 2, further comprising a follower attached to the sheath, the follower drivable relative to the sheath splitter assembly in a direction away from the proximal end of the delivery device assembly,
wherein movement of the follower in a direction away from the proximal end of the delivery device assembly slides the sheath over the sealing surface and pulls the prosthesis sheathing portion of the sheath from the covering position in which it covers the prosthesis receiving zone to the retracted position in which the prosthesis sheathing portion is longitudinally displaced way from the prosthesis receiving zone.

7. The endograft delivery device assembly of claim 6, wherein the follower is operatively attached to the sheath at a sheath distal end, and wherein the follower is sandwiched between a pair of spaced apart sheath portions.

8. The endograft delivery device assembly of claim 1, comprising a stiffener disposed around the guide wire cannula, the stiffener extending distally from the splitter body.

9. The endograft delivery device assembly of claim 8, wherein the stiffener is supported at a distal end thereof by the handle assembly.

10. The endograft delivery device assembly of claim 1, wherein the at least one splitter arm includes a side access passage into the through-hole of the splitter body.

11. The endograft delivery device assembly of claim 10, wherein the side access passage includes a side seal, the side seal configured to sealably receive a cannula.

12. The endograft delivery device assembly of claim 11, wherein the handle assembly comprises a main grip portion and the side access passage(s) extend out through the main grip portion of the handle assembly, thereby allowing cannulation of side fenestrations within an endograft.

13. The endograft delivery device assembly of claim 10, wherein the at least one splitter arm, presents an angled edge, with respect to a plane orthogonal to the positioner, to the sheath.

14. An endograft delivery device assembly comprising:
a proximal end;
a distal end;
a prosthesis receiving zone;
a tip at the proximal end;
a handle assembly at the distal end;
a guide wire cannula extending distally from the tip to the handle assembly, the guide wire cannula configured to slidably receive a guide wire;
a multi lumen positioner disposed around the guide wire cannula, the multi lumen positioner extending from the handle assembly towards the tip, wherein the handle assembly is configured to accommodate the multi lumen positioner;
a sheath extending from the handle assembly over the positioner towards the tip, the sheath having an inner surface, wherein the sheath includes a prosthesis sheathing portion, the prosthesis sheathing portion moveable from a covering position in which it covers the prosthesis receiving zone and a retracted position in which the prosthesis sheathing portion is longitudinally displaced way from the prosthesis receiving zone;
a sheath splitter assembly within the handle assembly, the sheath splitter assembly including:
a splitter body attached to the positioner and disposed within the sheath, the splitter body defining a through-hole through which the guide wire cannula passes; and
at least one splitter arm, the at least one splitter arm extending from the splitter body though a slit though the sheath to a support within the handle assembly, wherein the at least one splitter arm presents an angled edge, with respect to a plane orthogonal to the positioner, to the sheath;
wherein the splitter body includes a circumferential sealing surface extending towards the tip, the sealing surface and the inner surface of the sheath dimensioned such that the sheath is circumferentially stretched over the sealing surface thereby forming a seal.

15. The endograft delivery assembly of claim 14, wherein the splitter edges are configured to assist in splitting the sheath.

16. The endograft delivery assembly of claim 14, further comprising splitter arm mounts holding the at least one splitter arm in position relative to the handle assembly.

17. The endograft delivery assembly of claim 14, wherein the at least one splitter arm comprises a side seal disposed within the at least one splitter arms, the side seal configured to prevent blood loss through the positioner.

18. The endograft delivery device assembly of claim 14, further comprising a follower attached to the sheath, the follower drivable relative to the sheath splitter assembly in a direction away from the proximal end of the delivery device assembly,
wherein movement of the follower in a direction away from the proximal end of the delivery device assembly slides the sheath over the sealing surface and pulls the prosthesis sheathing portion of the sheath from the covering position in which it covers the prosthesis receiving zone to the retracted position in which the prosthesis sheathing portion is longitudinally displaced way from the prosthesis receiving zone.

19. The endograft delivery device assembly of claim 18, wherein the follower is operatively attached to the sheath at a sheath distal end, and wherein the follower is sandwiched between a pair of spaced apart sheath portions.

20. An endograft delivery device assembly comprising:
a proximal end;
a distal end;
a prosthesis receiving zone;
a tip at the proximal end;
a handle assembly at the distal end;
a guide wire cannula extending distally from the tip to the handle assembly, the guide wire cannula configured to slidably receive a guide wire;
a positioner disposed around the guide wire cannula, the positioner extending from the handle assembly towards the tip;
a sheath extending from the handle assembly over the positioner towards the tip, the sheath having an inner surface, wherein the sheath includes a prosthesis sheathing portion, the prosthesis sheathing portion moveable from a covering position in which it covers the prosthesis receiving zone and a retracted position in which the prosthesis sheathing portion is longitudinally displaced way from the prosthesis receiving zone;
a sheath splitter assembly within the handle assembly, the sheath splitter assembly including:
a splitter body attached to the positioner and disposed within the sheath, the splitter body defining a through-hole through which the guide wire cannula passes; and
at least one splitter arm having a side access passage into the through-hole of the splitter body, the at least one splitter arm extending from the splitter body though a slit though the sheath to a support within the handle assembly;
wherein the splitter body includes a circumferential sealing surface extending towards the tip, the sealing surface and the inner surface of the sheath dimensioned such that the sheath is circumferentially stretched over the sealing surface thereby forming a seal;
wherein the at least one splitter arm comprises a side seal disposed within the at least one splitter arm, the side seal configured to prevent blood loss through the positioner; and
wherein the sheath is configured to be pulled past the side access passage without breaking a hemostatic seal.
